SEQUENCE LISTING (21 sequences)

SEQ ID NO: 1 - DNA, 29903 bp, Human
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggttttgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttttaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggatgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacttacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtttctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - protein, 7096 aa, human
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGGAYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGGAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGGKIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQSKMSDVKCTSVVLLSVLQQLRVESSSKLWAQCVQLHNDILLAKDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQAGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPLTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQSLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQSSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 3 - protein, 1273 aa, human
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 419 aa, Human
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - protein, 75 aa, human
MYSFVSEETGTLIVNSVLLFLAFVVFLLVTLAILTALRLCAYCCNIVNVSLVKPSFYVYSRVKNLNSSRVPDLLV

SEQ ID NO: 6 - protein, 222 aa, human
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 7 - protein, 180 aa, human
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGG

SEQ ID NO: 8 - protein, 638 aa, human
AYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGG

SEQ ID NO: 9 - protein, 1945 aa, human
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 10 - protein, 500 aa, human
KIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQ

SEQ ID NO: 11 - protein, 305 aa, human
GFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQ

SEQ ID NO: 12 - protein, 290 aa, human
SAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQ

SEQ ID NO: 13 - protein, 83 aa, human
SKMSDVKCTSVVLLSVLQQLRVESSSKLWAQCVQLHNDILLAKDTTEAFEKMVSLLSVLLSMQGAVDINKLCEEMLDNRATLQ

SEQ ID NO: 14 - protein, 198 aa, human
AIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 15 - protein, 113 aa, human
NNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQ

SEQ ID NO: 16 - protein, 139 aa, human
AGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQ

SEQ ID NO: 17 - protein, 932 aa, human
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 18 - protein, 601 aa, human
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQ

SEQ ID NO: 19 - protein, 527 aa, human
AENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

SEQ ID NO: 20 - protein, 346 aa, human
SLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQ

SEQ ID NO: 21 - protein, 298 aa, human
SSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

(19) United States
(12) Patent Application Publication
(10) Pub. No.: US 2023/0168244 A1
(43) Pub. Date: Jun. 1, 2023

(54) COMPOSITIONS AND SURFACE ACOUSTIC WAVE BASED METHODS FOR IDENTIFYING INFECTIOUS DISEASE

(71) Applicant: AVIANA MOLECULAR TECHNOLOGIES, LLC, Orlando, FL (US)

(72) Inventors: Vanaja V. Ragavan, Orlando, FL (US); Soumen Das, Orlando, FL (US)

(73) Assignee: AVIANA MOLECULAR TECHNOLOGIES, LLC, Orlando, FL (US)

(21) Appl. No.: 17/921,862

(22) PCT Filed: Apr. 16, 2021
(86) PCT No.: PCT/US2021/027612

§ 371 Date: Oct. 27, 2022

Related U.S. Application Data
(60) Provisional application No. 63/016,851, filed on Apr. 28, 2020.

(51) Int. Cl. G01N 33/543; G01N 33/569

(52) U.S. Cl. CPC G01N 33/54373; G01N 33/56911; G01N 33/56983; G01N 2333/165; G01N 2333/20; G01N 2469/20

(57) ABSTRACT

The disclosure relates to systems and devices for diagnosing infectious disease (e.g., bacterial or viral infections). More particularly, the disclosure relates to acoustic sensors for detecting infectious disease caused by viral infections (e.g., coronavirus, rhinovirus, influenza, etc.).

### CROSS REFERENCE TO RELATED APPLICATIONS

This application is a national stage application, filed under 35 U.S.C. § 371, of International Application No. PCT/US2021/027612, filed Apr. 16, 2021, which claims priority to and the benefit of U.S. Provisional Patent Application No. 63/016,851, filed on Apr. 28 2020, and titled, “COMPOSITIONS AND SURFACE ACOUSTIC WAVE BASED METHODS FOR IDENTIFYING INFECTIOUS DISEASE”, the contents of each of which are incorporated herein by reference in their entirety.

### FIELD OF THE DISCLOSURE

The disclosure relates to systems and devices for diagnosing infectious disease (e.g., bacterial, fungal, parasitic infections, viral infections, etc.). More particularly, the disclosure relates to acoustic sensors for detecting infectious disease caused by viral (e.g., coronavirus, rhinovirus, influenza, etc.).

### BACKGROUND OF THE DISCLOSURE

Pandemic outbreaks of highly infectious and virulent virus strains (e.g., MERS-COV, SARS-COV, SARS-COV-2, H1N1 influenza, Ebola, and the like) present a serious risk to human and animal health worldwide. For example, genetic reassortment between human and avian influenza viruses can cause antigenic shifts that create novel viral proteins (e.g., a novel hemagglutinin (HA) of avian origin) for which humans have no immunity. The global influenza pandemics of 1918, 1957 and 1968 were the result of such antigenic shifts. The recent viral outbreaks caused by avian influenza viruses (e.g., H1N1, H5N1, H7N7 and H9N2 subtypes) and coronaviruses (e.g., MERS-COV, SARS-COV, and SARS-COV-2), and their infection of humans have created a new awareness of the pandemic potential of viral outbreaks. Globally, the 2020 SARS-COV-2 pandemic has already infected over 2.5 million people and killed almost 200,000. The estimated economic impact of the SARS-COV-2 pandemic is beyond the current ability to estimate, as the virus has shut down the global economy. In order to begin to manage the SARS-COV-2 pandemic and to begin the process of restarting the global economy, it is of paramount importance to develop rapid and accurate ways to test for viral infections. There is an urgent need for compositions and methods for identifying infections disease such as MERS-COV, SARS-COV, SARS-COV-2, H1N1 influenza, Ebola, and the like.

### SUMMARY OF THE DISCLOSURE

The disclosure relates to systems and devices for diagnosing infectious disease (e.g., bacterial, fungal, parasitic infections, viral infections, etc.). More particularly, the disclosure relates to acoustic sensors for detecting infectious disease caused by viral (e.g., coronavirus, rhinovirus, influenza, etc.), infections.

Other features and advantages of the disclosure will be apparent from the detailed description, and from the claims.

### BRIEF DESCRIPTION OF THE DRAWINGS

The present disclosure will now be described more fully hereinafter with reference to the accompanying drawings, in which preferred embodiments of the disclosure are shown. This disclosure may, however, be embodied in many different forms and should not be construed as limited to the embodiments set forth herein. Rather, these embodiments are provided so that this disclosure will be thorough and complete, and will fully convey the scope of the disclosure to those skilled in the art. Like numbers refer to like elements throughout. Other objects, features and advantages of the present disclosure will become apparent from the detailed description of the disclosure, which follows when considered in light of the accompanying drawings in which:

FIG. 1 shows different example configurations of the SSN antigen for COVID19 diagnosis. SSNs is a multiepitope antigen which consists of full-length S and N protein or receptor binding protein of S-protein and full length N protein, S1 and/or S2 subunit of S protein and full length of N-protein, or S1 subunit and full length or N-protein or S2 subunit of S protein and full length N-protein of SARS-Cov-2 virus. Other combinations consist of full length or subunits of Non-Structural Proteins 1-16, E, and/or M proteins in combination with full length or subunits of S and N proteins.

FIG. 2 is a schematic representation of the bio-coating developed with an antigen as a preferred recombinant antigen as an epitope of a Lyme disease Borrelia species for selective capturing Bb specific IgG and IgM or both.

FIG. 3 is a phase shift diagram from an example sensor with IgG and IgM positive plasma samples using the example SAW sensor having the immobilized recombinant antigen.

FIG. 4 is a fragmentary diagram showing examples of affinity bases strategies for the capture and enhanced sensitivity of Lyme disease detection by mass amplification on a SAW device.

FIG. 5 is a phase shift diagram from an example sensor with secondary anti-IgG antibody cross absorbed with human IgM and IgA.

### DETAILED DESCRIPTION OF THE DISCLOSURE

The disclosure features compositions and methods that are useful for the diagnosis, treatment and prevention of infectious disease, as well as for characterizing the infectious disease to determine a subject's prognosis and aid in treatment selection. The present disclosure is based, at least in part, on the discovery that recombinant, multipartite or multiepitope proteins may be engineered and covalently attached to the sensor surface of any testing device which uses an antigen/antibody binding event. Such a binding could also be reversed, whereby an antibody selective for these recombinant proteins can be placed on the testing device and the antigen thus detected from a biological sample to determine virus presence via its antigen detection. Described herein is an acoustic detection device (e.g., a Surface Acoustic Wave (SAW) device or a Bulk Acoustic Wave (BAW) device) to provide extremely sensitive detection of infectious disease-related antibodies (e.g., MERS-COV, SARS-COV, SARS-COV-2, H1N1 influenza, Ebola, and the like) in a sample. For example, recombinant, multiepitope proteins may include regions from more than one protein associated with SARS-COV-2 including, but not limited to, full-length S-protein linked to full-length N-protein, or the receptor binding domain of S-protein (aa 319-541) linked to the full length N-protein, or the 51 and/or S2 subunit of the S-protein linked to the full length N-protein, or 51 subunit of the S-protein linked to the full length N-protein, or the S2 subunit of S-protein linked to the full length N-protein of SARS-Cov-2. In some embodiments, it is contemplated that different epitopes in the recombinant protein may be separated by an amino acid spacer or linker (e.g., a 3-20 amino acid linker). These proteins can be formed by introducing the appropriate genetic material into growing cells, from which the expressed protein or proteins of interest may then be isolated. The epitopes on these proteins may be retained for binding purposes. In some embodiments, the different epitopes in the recombinant protein may not be separated by an amino acid spacer or linker. According to the techniques herein, the sensor surface may capture specific IgG and IgM antibodies present in infected patient plasma samples. According to the techniques herein, recombinant technology may be used to prepare different combinations of antigenic epitopes to produce a series of SSN-antigens for diagnosis of a variety of virally induced infectious diseases (e.g., MERS-COV, SARS-COV, SARS-COV-2, H1N1 influenza, Ebola, and the like). The techniques herein provide serological detection, as well as detection of virus particles, virus proteins, and the like. For example, an antibody selective for these viral proteins or viral particles may be placed on the sensor surface of the testing device to detect an antigen within a biological sample, which in turn may detect an infectious agent (e.g., a bacterial pathogen, virus, etc.). The active binding agent (e.g., antibody, or viral proteins or chimeric proteins) is adhered to the sensor surface via covalent interactions. The target molecules (e.g., antibody or virus particles or viral protein) may bind with the active binding agent covalently bound to the surface of the sensor, and the binding occurs at the level of the sensor surface. Specific binding of the target biologic molecules causes alterations in mass/viscosity that change the pattern of acoustic transmission by the sensor surface, thereby allowing detection of the target biologic molecules.

It is also contemplated within the scope of the disclosure that the techniques herein may be applied to non-virally induced infectious disease such as Lyme disease, or other diseases wherein a recombinant protein or antibody is generated against the disease-causing microorganism and can be used to detect the presence of an infection or infectious agend. For example, another recombinant antigen “DOC” which consists of full-length DbpA, PepC10, and C6 from the pathogen that causes Lyme disease, Borrelia (Bb) burgdorferi, may be covalently bound to a SAW or BAW sensor surface and used to capture Borrelia (Bb) burgdorferi-specific IgG and IgM antibodies present in infected patient plasma samples with Lyme disease. The techniques herein further may be utilized for other types of detection systems, including both acoustic (SAW, BAW, Rayleigh wave, and the like) and other optical and electrochemical detection systems (e.g., Surface Plasmon Resonance, ELISA, and the like). The present disclosure therefore provides point of care compositions and methods for improved detection of host generated antibodies against viral and bacterial infectious disease.

Conventional diagnostic methods for characterizing infectious disease (e.g., MERS-COV, SARS-COV, SARS-COV-2, H1N1 influenza, Ebola, Lyme disease, and the like) are not quantifiable or high throughput, notoriously unreliable and lack reproducibility.

Accordingly, the disclosure provides improved diagnostic compositions that are useful for identifying subjects or biological samples as having viral infection (e.g., MERS-COV, SARS-COV, SARS-COV-2, H1N1 influenza, Ebola, and the like). The disclosure further provides compositions and methods for identifying subjects or biological samples as having bacterial infections (e.g., Lyme disease, etc.). The disclosure further provides methods of using these compositions to identify a subject's prognosis, select a treatment regimen, and monitor the subject before, during or after treatment.

### SARS-Cov-2

Severe acute respiratory disease corona virus (SARS-Cov)-2 was first reported in Wuhan, China on Dec. 31, 2019. SARS-Cov-2 is a β-coronavirus closely related to the coronavirus SARS-Cov-1 isolated in 2002-2003 from bats. SARS-Cov-2 causes the Coronavirus disease 2019, or COVID19. The virus has infected more than 560,000 people in the United States and has resulted in more than 22,000 deaths. The global number of infected cases is reported to be at least 1.85 million, with more than 114,000 reported fatalities at the time of the instant disclosure. Therefore, a quick, efficient and low-cost point of care (POC) diagnostic test is needed. Efficient and low-cost point of care (POC) diagnostics enable positive individuals to be tracked and isolated, thereby controlling the spread of the virus. Nucleic acid tests are the extant diagnostic test for SARS-Cov-2 and COVID19 diagnosis. Clinical trials for nucleic acid-based diagnostics showed high sensitivities of over 90% for positive samples. However, real clinical settings report the sensitivity to not be nearly as high. Many nucleic acid tests exhibited apparent false negatives in patients exhibiting clinical symptoms of COVID19 or in imaging studies consistent with pneumonia. The false negatives associated with nucleic acid tests might be due to the clinical specimen used, the sample collection and/or the extraction procedure (Pan Y et al, 2020, Clinical Chemistry). A recent publication revealed that nucleic acid test using nasal swab (n=8) and sputum (n=104) samples from COVID19 patients exhibited only 63% and 75% accuracy in positive tests, respectively (Wang W et al., 2020, JAMA). Therefore, there is a vital world-wide need for viral tests that can accurately quantify SARS-Cov-2 exposure. In one embodiment of the instant disclosure, attachment of specific antibodies to a sensor surface to detect virus or viral antigens of SARS-CoV-2 using acoustic waves is a rapid and accurate measure of presence of virus. These antibodies can be made against the recombinant proteins thus synthesized with a high affinity diagnosis as described below to detect the various antigens or surface proteins/particles of a virus such as SARS-CoV-2.

Extant serology antibody testing has provided key information into the incidence and prevalence of previous COVID 19 exposure in the population. Serology antibody-based tests can identify individuals who have been exposed to SARS-Cov-2 and who have developed antibodies to the virus, but who are either no longer symptomatic or who present as asymptomatic. A detectable titer of an antibody to SARS-Cov-2 viral plasma membrane proteins is detectable in COVID19 patients from day 6 onwards after exposure. However, current serological tests lack efficacy because of weak interactions with the virus and/or fluctuating immunity in the subject. Two major proteins, nucleocapsid protein (N-protein) and spike protein (S-protein) are encoded by all β-coronaviruses, including SARS-Cov-2. The Enzyme-linked Immune Sorbent Assay (ELISA) or POC test uses either recombinant N-protein or recombinant S-protein to capture IgG and IgM antibodies generated by COVID19 patients. The N-protein is more immunogenic compared to the S protein. However, the SARS-Cov-2 N-protein may bind to antibodies against other β-coronaviruses, making a test based on the N-protein less specific. The S-protein has exhibited its own challenges, wherein POC and ELISA tests have indicated that the low titer of SARS-Cov-2 S protein antibody-based tests are less sensitive because of the low titer of S protein antibodies (Amanat F et al, 2020, medRiv; Haveri A, et al, 2020, Euro Surveill). The non-structural proteins (NSPs), as well as the E and/or M proteins of SARS-Cov-2 virus may be immunogenic as well. The NSPs 1-16 comprise a 3C-like proteinase, an RNA-dependent RNA polymerase, a helicase, a 3′-to-5′ exonuclease, and endoRNAase, and a 2′-O-ribose methyltransferase. In some embodiments of the instant disclosure, NSPs 1-16, E, and/or M proteins of SARS-Cov-2 may be used in combination with S and N proteins to identify SARS-Cov-2 antibodies in serum samples. Accurate and rapid POC diagnosis remains one of the greatest obstacles to the clinical management of COVID19 patients. At the time of the instant disclosure, extant methods of POC and ELISA based CODVID19 diagnostic tests have faced significant challenges in terms of both their specificity and sensitivity.

Notably, the instant disclosure provides acoustic wave-based antigen/antibody testing. In one embodiment, Surface Acoustic Wave (SAW) which is based on an innovative, fast (<10 min), cost-effective, and robust POC diagnostic platform, is adapted as a COVID19 diagnostic. While most detection technologies used to diagnose biological phenomenon traditionally employed light and electro chemical sensors, recent advances in acoustic technologies have allowed for the use of acoustic methods for biological sensing. Acoustic methods utilize a responsive piezoelectric material that responds to an electrical signal by creating an acoustic wave (i.e., very high frequency sound) as the fundamental sensing property. These SAW systems have not achieved widespread use as detection technologies because it is traditionally difficult to work in liquid environment and bind biologically capture agents onto the surface of acoustically transmissive materials. The crystals involved on the surface of acoustically transmissive materials, namely, quartz, lithium niobate and tantalate, etc., are typically only weakly responsive to the adhesion of biological materials. Some chemical agents have been adapted to provide adhesion of biological molecules on these crystals, such as silane compounds with reactive amine residues. The approach has been to decorate the surface of the crystal with a metal which is readily amenable to the attachment of biologicals. In this regard, the most used surface metal continues to be gold (e.g., McBride & Cooper 2008). Relative to gold, other metals, including aluminum, bind biologicals poorly, hence their reported use in SAW devices is minimal. In contrast, aluminum surfaces propagate acoustic waves more effectively but, as noted above, have been difficult to work with in biological applications. Aluminum may be laid onto the surface of the sensor during fabrication to serve as a critical waveguide. Another approach has been to use a “Love layer” composed of for example, but not limited to, a polymer, a ceramic such as SiO2, Poly (methyl methacrylate), or gold, on the surface of the SAW sensor which concentrates the energy of the acoustic wave closer to the surface for effective analyte detection.

In some embodiments of the instant invention, a novel method to adhere biomolecules to aluminum coated biosensors by the use of a linker is described. The instant disclosure describes methods that result in stable, robust, covalently bound surface coatings of the aluminum (or similarly, of other metals). These coatings retain functional anchored biomolecules including but not restricted to proteins, antibody, nucleic acid and small molecules with a primary amine. In addition, the method of immobilizing biomolecule improves the sensitivity of the sensor when combined with sensitive electrical systems, such as SAW. The methods described in this application result in covalently bound affinity capture agents including but not restricted to antibodies, variable fragment of antibody, protein antigens, nucleic acid, aptamers or other such molecules on the SAW sensor for the selective capturing of a target analyte. It is critical that surface adhesion results in the proper orientation of the said affinity agents on the aluminum surface to selectively and specifically capture the analyte of interest. These activated moieties may or may not be used with a linker such as disuccinimidyl suberate (DSS) for covalent conjugation and minimize steric hindrance. Biological agents utilized here that are known to be bioactive include molecules with amine group/s including proteins, polymers and nucleic acid entities. Various methods have been used for activation of the surface of the SAW sensors, including heat, radiation and gases such as oxygen or nitrogen. These different processes offer a range of treatments under multiple conditions. The aluminum surfaces of the SAW sensors are activated under these conditions resulting in the enhanced covalent binding of biologically active capture reagents. The combination of surface modification and biomaterials serves as a universal platform to decorate the surface of SAW sensors with any antigen (protein), antibody or other affinity capture agents for the specific capture of desired target molecules.

In particular for viral or bacterial infectious disease (e.g., SARS-COV-2, Influenza, Lyme Disease, and the like) diagnosis, one embodiment of the instant disclosure employs a recombinant protein for serological diagnosis using acoustic sensors such as using SAW. In some embodiments, SARS-COV-2 S-protein, or parts thereof, may be combined with full length, or parts thereof, of any of the following SARS-COV-2 proteins: N-protein, E-protein, M-protein, and NSP1, NSP2, NSP3, NSP4, NSPS, NSP6, NSP7, NSP8, NSP9, NSP10, NSP12, NSP13, NSP14, NSP15, and NSP16. Linkers between parts of the recombinant protein may include for example, but not limited to, Serine and Glycine in combinations of 3-20 amino acids, glycine repeats of 8-10 amino acids, and rigid linkers such as, for example, (EAAAK)ₙ where n=1-3.

In some embodiments, SARS-COV-2 N-protein, or parts thereof, may be combined with full length, or parts thereof, of any of the following SARS-COV-2 proteins: S-protein, E-protein, M-protein, and NSP1, NSP2, NSP3, NSP4, NSPS, NSP6, NSP7, NSP8, NSP9, NSP10, NSP12, NSP13, NSP14, NSP15, and NSP16. Linkers between parts of the recombinant protein may include for example, but are not limited to, Serine and Glycine in combinations of 3-20 amino acids, Glycine repeats of 8-10 amino acids, and rigid linkers such as, for example, (EAAAK)ₙ where n=1-3.

In some embodiments, SARS-COV-2 E-protein, or parts thereof, may be combined with full length, or parts thereof, of any of the following SARS-COV-2 proteins: N-protein, S-protein, M-protein, and NSP1, NSP2, NSP3, NSP4, NSP5, NSP6, NSP7, NSP8, NSP9, NSP10, NSP12, NSP13, NSP14, NSP15, and NSP16. Linkers between parts of the recombinant protein may include for example, but not limited to, Serine and Glycine in combinations of 3-20 amino acids, Glycine repeats of 8-10 amino acids, and rigid linkers of (EAAAK)ₙ where n=1-3.

In some embodiments, SARS-COV-2 M-protein, or parts thereof, may be combined with full length, or parts thereof, of any of the following SARS-COV-2 proteins: N-protein, E-protein, S-protein, and NSP1, NSP2, NSP3, NSP4, NSP5, NSP6, NSP7, NSP8, NSP9, NSP10, NSP12, NSP13, NSP14, NSP15, and NSP16. Linkers between parts of the recombinant protein may include for example, but are not limited to, Serine and Glycine in combinations of 3-20 amino acids, Glycine repeats of 8-10 amino acids, and rigid linkers such as, for example, (EAAAK)ₙ where n=1-3

In some embodiments, serological identification using sera known to contain antibodies was 100% accurate, which provided a higher standard than western blots or ELISA detection.

While most detection technologies used to diagnose biological phenomenon have traditionally employed optical and/or electro-chemical sensors, recent advances in acoustic technologies now allow for the potential use of acoustic methods for biological sensing. Acoustic methods utilize the function of a responsive piezoelectric material that reacts to an electrical signal with the creation of an acoustic wave (i.e., very high frequency sound) as the fundamental sensing property. Extant SAW systems did not achieve widespread use as detection technologies because they were difficult to work within liquid environments where it was necessary to bind any capture agents onto the surface of largely non-reactive acoustically transmissive materials. The crystals involved, namely, quartz and similar materials, such as lithium niobate and tantalite and similar materials were typically only weakly, if at all, responsive to the adhesion of biological materials.

To overcome this issue, chemical agents were used to provide adhesion of biological molecules onto these crystals, such as silane compounds with reactive amine residues. One approach was to decorate the surface of the crystal with a metal, which is readily amenable to the attachment of biologicals. For example, the most commonly used surface metal is gold, as described in the article by McBride and Cooper, “A High Sensitivity Assay for the Inflammatory Marker C-Reactive Protein Exploring Biosensing,” Journal of Nanobiotechnology, 2008, the disclosure which is hereby incorporated by reference in its entirety. Relative to gold, other metals, including aluminum, bind biologicals poorly, hence their published use in SAW devices used in biotechnological applications has been minimal. On the other hand, aluminum surfaces propagate acoustic waves very effectively, but because their ability to adhere biological materials are poor as noted above, their use in biotechnological applications was limited. The sensors described in the above-identified provisional applications may include aluminum as waveguide, which is fabricated onto the surface of the sensor.

A number of applications have been filed relating to the biosensors, including different coatings, layers, signal amplification, interfaces with fluid materials, and multiplexing, such as disclosed in U.S. provisional patent application Ser. No. 62/529,986 filed Jul. 7, 2017, entitled “BIOACTIVE COATING FOR SURFACE ACOUSTIC WAVE SENSOR”; U.S. provisional patent application Ser. No. 62/529,945 filed Jul. 7, 2017, entitled “METHODS AND APPARATUS FOR INTERFACING SENSORS WITH FLUID MATERIALS”; U.S. provisional patent application Ser. No. 62/529,725 filed Jul. 7, 2017, entitled “MULTIPLEXING SURFACE ACOUSTIC WAVE SENSORS WITH DELAY LINE CODING”; U.S. provisional patent application Ser. No. 62/530,735 filed Jul. 10, 2017, entitled “BIOACTIVE LAYER AND BIOSENSOR DEVICE CONTAINING THE SAME”; and U.S. provisional patent application Ser. No. 62/531,237 filed Jul. 11, 2017, entitled “SIGNAL AMPLIFICATION IN BIOSENSOR DEVICE,” the disclosures which are hereby incorporated by reference in their entirety.

For purposes of description, the '986, '945, '725, '735, and '237 provisional patent applications are summarized below to understand better the sensor technology using a recombinant antigen referred to the “DOC” antigen on the sensor surface for capturing Bb-specific IgG and IgM antibodies present in patient plasma samples such as taken from a finger stick derived blood sample.

Surface acoustic wave (“SAW”) based sensors perform biochemical sensing and analysis in liquid media. Different SAW devices are disclosed which include the shear-h horizontal SAW (“SH-SAW”), guided SH-SAW sensors (also called Love-Wave devices), and SAW sensors without a waveguide.

In this '945 application, a liquid cell may interface sensor elements with an introduced liquid media for biochemical analysis. The liquid cell can be configured to isolate the acoustic wave path and the sensor elements using air pockets, which may be created without using physical walls. In an example, the non-physical walls are air-liquid virtual walls. The sensor may include a substrate, at least one sensor unit, and a top layer. The sensor unit may include a sensor element, a pair of electrical components located on opposite ends of the one sensor element and at least one peripheral wall disposed on the substrate and configured to surround the pair of electrical components and at least a portion of the sensor element. The top layer may be disposed over the at least one peripheral wall and create an air pocket over each of the electrical components. In an example, the sensor may be a SAW sensor or a BAW sensor and may include a fluidic channel over a portion of the sensor element and configured to receive a liquid medium. The substrate may include a piezoelectric material.

The sensor element may include a modified substrate surface configured to capture at least one analyte. At least one of the pair of the electrical components may be an interdigital transducer and one of the pair of electrical components may include a reflector or at least one interdigital transducer. The sensor element and pair of electrical components may be aligned along an axis and the liquid media may be configured to enter the fluidic channel through an inlet on a first end of the fluidic channel and exit the fluidic channel through an outlet on a second end of the fluidic channel. At least one peripheral wall may be formed from any one of a plastic sheet, double-sided tape, injection molding material, and gasket. The air pocket over the electrical component may have a thickness of about 0.1 μm to about 1 μm.

In the '237 provisional application, signals may be amplified to the biosensor by applying a sample to the biosensor having a capture reagent that may be one or more first recognition moieties for binding an analyte. The capture reagent may be immobilized on the biosensor. A signal amplifying material is introduced, which may have one or more second recognition moieties for binding to the analyte. The presence or quantity of an analyte in a sample may be determined by applying a sample to the biosensor having a capture reagent having one or more first recognition sites for binding an analyte. The capture reagent may be immobilized on the biosensor and the signal amplifying material may be introduced. The polymer or metallic material may have one or more second recognition sites to bind the analyte in a different portion of the analyte and any change may be measured in amplitude, phase or frequency of a biosensor signal as a result of the analyte binding to the signal amplifying material. The biosensor component may include a piezoelectric substrate and a capture reagent that may be immobilized on the piezoelectric substrate. The capture reagent may have a first recognition site for an analyte and the signal amplifying material may have a second recognition site for the analyte.

In the '735 provisional application, a biosensor component includes a piezoelectric substrate and a capturing reagent immobilized on the piezoelectric substrate. In an example, the substrate may include a three-dimensional (3D) matrix microstructure configured to increase the number of capturing reagents immobilized on the piezoelectric substrate. Capturing reagents may be immobilized on the piezoelectric substrate through binding to the 3D matrix microstructure. A biosensor component may be fabricated by forming a 3D matrix microstructure on a piezoelectric substrate to increase the surface area of the piezoelectric substrate and immobilizing one or more capturing reagents on the piezoelectric substrate.

Also, the presence or quantity of an analyte in a sample may be determined by contacting the biosensor component with the sample and generating an acoustic wave or bulk wave across the metal or plain substrate. Any change in amplitude, phase or frequency of the acoustical or bulk wave may be measured as a result of the analyte binding to the capture reagent. Polymers of poly (methyl methacrylate) (PMMA) as a Love Wave plasma etching to create a 3D structure on the surface of the sensor may increase the surface area.

In the '986 provisional application, a biosensor component includes a substrate coated with a metal and an anchor substance that includes a binding protein or nucleotide and a functional group having at least one sulfur atom. The anchor substance binds directly to the metal through the functional group and forms a monolayer on the metal. The anchor substance is configured to couple to a capture reagent.

A surface of a metal material and/or plain crystal surface may be coated with a bioactive film by applying a first composition as an anchor substance to the surface of the metal/crystal material to form a monolayer on the surface. The anchor substance includes a binding protein in a functional group having at least one sulfur. A second composition may be applied as a biotinylated capture reagent to the monolayer of the anchor substance. The biotinylated capture reagent binds to the anchor substance through the binding protein to form a layer of the biotinylated capture reagent.

Biosensor components may include a piezoelectric substrate and an anchor substance bound to a surface of the piezoelectric substrate. The anchor substance may include a spacer and a binding component and a capture reagent. The anchor substance may be coupled with the capture reagent through the binding component.

It is possible to coat a surface of the piezoelectric material of a biofilm by applying a first composition including an anchor substance to the surface of the metal/crystal material to form a monolayer on the surface. This anchor substance includes a spacer coupled to a binding component. A second composition as a biotinylated capture reagent may be applied to the monolayer of the anchor substance. The biotinylated capture reagent may bind to the anchor substance through the binding component of the anchor substance to form a layer of the biotinylated capture reagent.

Other embodiments relate to determining the presence or quantity of an analyte in a sample by contacting the biosensor component with the sample and generating an acoustical or bulk wave across the coated substance and measuring any change in amplitude, phase or frequency of the acoustic/bulk wave as a result of the analyte binding to the capture reagent. Other embodiments relate to a bulk wave resonator as the biosensor component described in this '986 patent application.

In the '725 provisional application, a multiplexing SAW measurement system determines a variance in at least one of amplitude, phase, frequency, or time-delay between pulses of the receiving signal (Rx) and/or the excitation signal. The multiplexing SAW measurement system can include phase detection which determines a phase corresponding to each of a plurality of pulses with respect to each other and/or the excitation signal. The difference in delay line length between the SAW sensors results in a time delay between the pulses of the received signal (Rx). The shifts in time domain between the pulses of the compressed pulse train correspond to phase shifts associated with a particular SAW sensor. These phase shifts can be determined, for example, using a software program or FPGA (field programmable gate array) hardware.

The SAW device may include a piezoelectric substrate and a plurality of SAW sensors attached to the piezoelectric substrate and arranged on its surface, and in an example, may include a first SAW device and a second SAW device. The first SAW sensor may include a first delay line configured to propagate a first surface acoustic wave. The second SAW sensor may include a second delay line configured to propagate a second surface acoustic wave. A length of the first delay line may be greater than a length of the second delay line or the length of the second delay line may be greater than the length of the first delay line. The first SAW sensor may further include a first transducer for transmitting the first surface acoustic wave along the first delay line and a second transducer for receiving the first surface acoustic wave upon propagation of the first surface acoustic wave along the first delay line. The first SAW sensor may further include a transducer positioned on the substrate and a reflector positioned on the substrate opposite the transducer, which may be configured to transmit the first surface acoustic wave along the first delay line.

The transducer may be configured to receive the first surface acoustic wave after the first surface acoustic reflects off the reflector and propagates along the first delay line twice. The reflector may be a first reflector and the first SAW sensor may further include a second reflector positioned on the substrate proximate the first reflector relative to the transducer. The transducer may be configured to receive the first surface acoustic wave upon reflecting off the second reflector and propagating along the first delay line twice. The first reflector may be configured to reflect a surface acoustic wave having a first frequency and the second reflector is configured to reflect a surface acoustic wave having a second frequency.

The first SAW sensor may include a first pair of electrical contacts and the second SAW sensor may include a second pair of electrical contacts. The first and second pairs of electrical contacts are electrically connected. Each of the SAW sensors may be configured to receive an excitation signal. The excitation signal may include at least one of a pulse voltage, a sinusoidal electrical signal, frequency modulation, linear frequency modulation, hyperbolic frequency modulation, orthogonal frequency coding, random modulation, continuous phase modulation, frequency shift key, multi-frequency shift key, phase shift key, wavelet modulation, or a wideband frequency signal. Each of the SAW sensors may be configured to simultaneously receive the excitation signal. The SAW device may further include one or more processors in communication with each of the first SAW sensor and the second SAW sensor. The processors may be configured to generate a receiving signal based at least in part on signals received from the first SAW sensor and the second SAW sensor. The one or more processors may be further configured to determine or monitor at least one analyte based at least in part on the receiving signal and may identify the at least one analyte by detecting a variance in amplitude, phase, frequency, or time-delay between at least two of a pulse corresponding to the excitation signal, a pulse corresponding to the first SAW sensor, or a pulse correspond to the second SAW sensor.

The receiving signal may include a compressed pulse train having a plurality of pulses and include a first pulse corresponding to the first SAW sensor and a second pulse corresponding to the second SAW sensor. A timing of the first pulse is based at least in part on the length of the first delay line, and a timing of the second pulse is based at least in part on the length of the second delay line. The plurality of pulses of the compressed pulse train may include a pulse corresponding to the excitation signal. The piezoelectric substrate may include at least one of 36° Y quartz, 36° YX lithium tantalite, langasite, langatate, langanite, lead zirconate titanate, cadmium sulfide, berlinite, lithium iodate, lithium tetraborate, or bismuth germanium oxide. The piezoelectric substrate may include a piezoelectric crystal layer and include a thickness greater than a Love Wave penetration depth on a non-piezoelectric substrate.

The SAW device may further include a sensing region located at the first delay line and configured to attach to or react with an analyte. The sensing region may include a biologically sensitive interface for capturing analytes from a liquid media. The sensing region may include a chemically sensitive interface for absorbing analytes from a liquid media.

The SAW device may further include a detector for measuring a phase response of surface acoustic waves as a function of an analyte added to the sensing region and a guiding layer on the first delay line. The guiding layer may include at least one of a polymer, SiO₂ or ZnO. A first surface acoustic wave may correspond to the first SAW sensor and include a frequency greater than 100 MHz, greater than 300 MHz, greater than 500 MHz, or greater than 1000 MHz.

Lyme Disease

Lyme disease (LD) is an infectious and potentially post-infectious inflammatory disease caused by Borrelia burgdorferi (Bb) in the United States and other species around the rest of the world and transmitted through an infected tick bite. Typically, Lyme disease is transmitted from a bite of an infected tick of the Ixodes genus. Although Bb is the primary bacteria causing the disease, other species such as Borrelia mayonii in the United States and Borrelia afzelii and Borrelia garinii in Europe and Asia cause the disease. Possible other species may include Borrelia bissettii and Borrelia valaisiana.

According to the World Health Organization, Lyme disease is now the most prevalent vector-borne disease in the northern hemisphere with greater than 3 million diagnostic tests performed per year in the United States. Unfortunately, a limited number of Bb spirochete in the infected blood restricts the direct detection of the antigen or Bb. To date, serology tests are typically more effective for screening clinically suspected cases of Lyme disease. Current standards typically rely on two separate and sequential (2-tier) tests, i.e., 1) ELISA followed by 2) immunoblot, which can routinely take multiple days to complete, require technical expertise, and be prone to subjectivity, which leads to potential misinterpretation. Therefore, accurate and rapid diagnosis remains one of the greatest obstacles to the clinical management of Lyme disease. As of now, there are no adequate point of care (POC) tests for diagnosing Lyme disease.

There have been some approaches to overcome the need to bind directly to aluminum by using silicone as a Love Layer. Biological materials may adhere directly to silicon dioxide rather than directly on aluminum, and this approach has been used previously such as described in the Sandia National Laboratories U.S. Pat. No. 8,709,791, the disclosure which is hereby incorporated by reference in its entirety. In that example, a lithium tantalite based SAW transducer includes a silicon dioxide waveguide sensor platform featuring three test and one reference delay lines to absorb antibodies directed against a Coxsackie virus B4 or a negative-stranded category A bioagent Sin Nombre virus (SNV). This Love Layer has an advantage because it can concentrate the energy of the acoustic wave closer to the surface for effective analyte detection. In an example, the Love Layer may be a polymer or ceramic such as SiO₂, poly (methyl methacrylate), gold or other materials. This type of system could be used in further sensor development.

The disclosure provides acoustic wave sensors that overcome the problems noted above with the binding of biological agents to aluminum. The current disclosure describes sensors that allow the adhesion of biomolecules to aluminum coated biosensors in a technique never previously accomplished. By the use of a linker, the sensors have a stable, robust, and more importantly, covalently bound surface coatings on the aluminum (or other metals) fabricated on these crystals. These coatings retain functional activity of the anchored biomolecules and as a non-limiting example, with an amine, such as a primary amine. In addition, the method of immobilizing biomolecules improves sensitivity of the sensor when combined with the electrical/acoustic system.

It has also been found that the techniques described below and developed by the inventors result in covalently bound affinity capture agents, including but not restricted to antibodies, variable fragments of an antibody, protein antigens, a nucleic acid, aptamers, lipids, lipoproteins or other such molecules on the acoustic sensors of any variety, including piezoelectric acoustic sensors such as SAW, Love Layer, Raleigh, BAW, and similar sensors for the selective capturing of a target analyte. It is also noteworthy that the surface adhesion results in the proper orientation of the affinity agents on the aluminum surface to capture selectively and specifically the analyte of interest. These activated moieties may or may not be used with a linker such as disuccinimidyl suberate (DSS) for covalent conjugation and minimize steric hindrance. Biological agents utilized here are known to be bioactive and include well known agents such as molecules with one or more amine groups, including proteins, polymers and nucleic acid entities.

As will be explained below, various techniques can be used for activating the surface of the SAW sensors, including the application of one or more of heat, radiation and gases such as oxygen or nitrogen. These different processes offer a range of treatments under multiple conditions. The aluminum surfaces of the SAW sensors could be activated under these conditions resulting in the enhanced covalent binding of biologically active capture reagents. The combination of surface modification and biomaterials serve as a universal platform to decorate the surface of SAW sensors with any antigen (protein), antibody or other affinity capture agents for the specific capture of desired target molecules. In particular for Lyme disease diagnosis, the sensor technology uses a covalently attached “DOC” antigen, which is a recombinant antigen that consists of full-length DbpA, PepC10, C6, and on the sensor surface for capturing Bb-specific IgG and IgM antibodies present in infected patient plasma samples.

An example of the recombinant antigen preparation and its use with different combinations of antigenic epitopes, including the “DOC” for Lyme diagnosis are disclosed in U.S. Patent Publication No. 2016/0237478 to Jewett et al., assigned to the University of Central Florida Research Foundation, Inc., the disclosure which is hereby incorporated by reference in its entirety. This '478 publication discloses an improved approach for detecting Lyme disease that uses the sensitivity of PCR combined with a detection strategy that uses a limited number of antigens for determination of host response antibodies. In one example, it uses an immune-PCR (iPCR), and optionally, employs a single hybrid recombinant antigen that combines three highly antigenic in vivo-expressed B. burgdorferi antigens for objective and highly sensitive and specific detection of a host immune response to B. burgdorferi infection. This hybrid recombinant antigen is designated in the application and also throughout this description as “DOC” and is a full length DBPA protein fused to the C6 peptide of VIsE and the PEP10 peptide of OspC. It discloses an iPCR method that includes aspects of a liquid-based protein detection method that combines the sensitivity of PCR with the specificity and versatility of immuno assay-based protocols. Thus, the iPCR approach is combined with a single hybrid antigen and a number of the challenging detection issues related to Lyme disease diagnostics are alleviated. Thus, with the sensors as disclosed, there is now a single streamlined quantitative test that may provide equivalent sensitivity and increased specificity compared to existing two-tier testing.

It should be understood that the recombinant antigen has an epitope of a Borrelia species and may include a protein or portion thereof having a sequence derived from Borrelia species. The recombinant antigens may include but not be limited to full length sequences or portions of the OspC, BmpA, VIsE, DbpA, BPK19, OspA, RevA, Crasp2, BBK50, or portions or combinations or fusions of the different proteins. Recombinant antigens may include a tag such as a GST tag, a hemagglutinin, or C-Myc or combinations. Other examples are listed throughout the incorporated by reference '478 publication.

The sensor platform as described in the incorporated by reference applications identified above may be modified to use the DOC antigen and may be a Point of Care (POC) technique for improved detection of host generated antibodies against Bb. This innovation can be extended to any piezoelectric based acoustic sensing including SAW, SAW, Raleigh and Love Waves as non-limiting examples. This innovation can also be extended to a variety of recombinant and chimeric proteins aimed at the antigens secreted by or found on Borrelia genus of any species under the above described conditions.

The sensor platform as described above in the incorporated by reference provisional applications is decorated with the DOC antigen or similar recombinant/chimeric antigens or mixture of antigens and can be used for Lyme disease diagnostic testing. For an efficient SAW biosensor, it is important to bind any antigens covalently in a proper orientation for binding with Bb specific antibodies from infected blood, serum or plasma. In an example, the surface of the SAW sensor developed by the assignee, Aviana Molecular Technology, is a metal or aluminum deposited on a crystal surface. Sections of the sensor also contain aluminum alternating with crystal. Various crystals can be used along with various crystal cuts. Nevertheless, the approaches for the use of SAW sensors for the detection of Bb specific antibodies are based on the ability to decorate the sensor surface with an appropriate antigen, as discussed above. For the detection of Bb specific antibodies (IgG and IgM), the sensor surface is decorated with the appropriate antigen material that can selectively capture the desired target Bb specific IgG, IgM or both, and in an example, the DOC antigen.

There now follows a description of a technique that can be used to attach the DOC antigen on the SAW based metal or crystal sensor surface as a capturing molecule. However, it should be understood that the approach is not limited to antigens and can be adapted to immobilize other capture agents that may include a primary amine group, including but not limited to a protein, protein fragments, an antibody, antibody fragments, aptamers or nucleotide fragments, or small molecules on the sensor surface. There also follows a description of a technique that specifically enhances the detection sensitivity of the sensor.

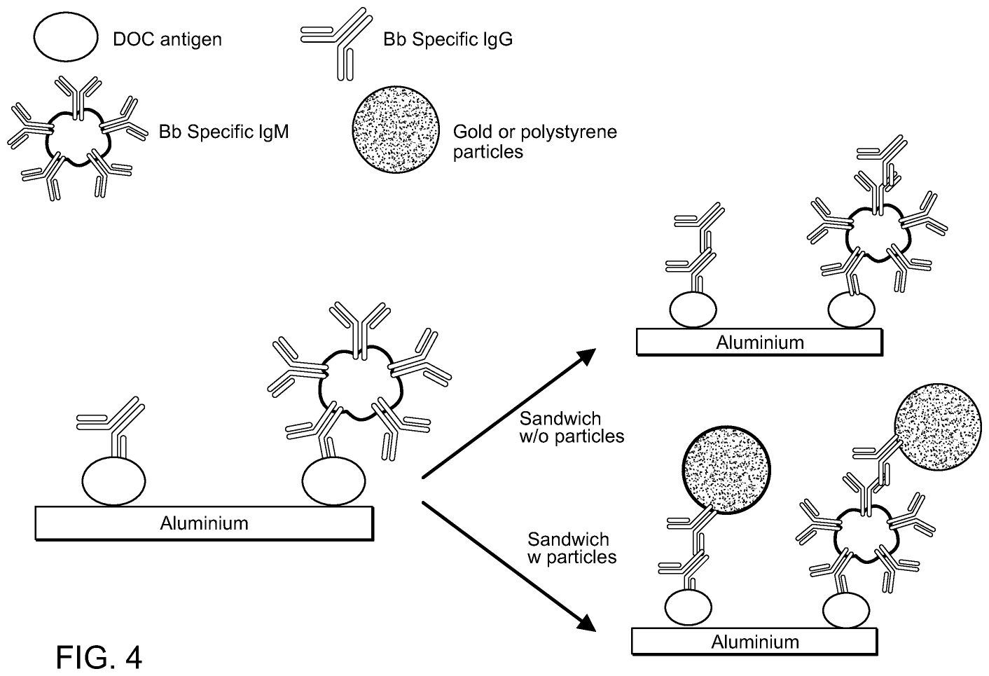

Referring now to FIG. 4, there is illustrated a schematic of a bio-coating developed for the DOC antigen for selectively capturing Bb specific IgG and IgM or both. The sequence is part of the technique for bio-coating the aluminum following surface activation and derivatization by covalent attachment of a biological capture agent as the DOC antigen in this case to the surface via a linker.

In this technique, the native aluminum and crystal surface were first activated by plasma or gaseous cleaning (minutes to hours). The exposure of the sensor to plasma cleaning creates hydrophilic functional groups on aluminum and crystal surfaces that can be readily measured by evaluating the contact angle. Contact angles significantly less than 90° are optimal for subsequent attachment of reagents to the activated surface. Afterward, the activated surface was subsequently coated with a silane having an amine functional group. The concentration of the silane is important to ensure the formation of a monolayer and depends on the reaction conditions. In an example, 0.25-20% of silane in an alcohol:water mixture having a pH of about 4 to 6 was used for the coating. This coating process was carried out for one minute to 1 hour. Next, disuccinimidyl suberate (DSS) as a linker molecule was covalently attached with the amine silane. This reaction was carried out at about a pH of 6 to 7.5 using a sodium phosphate-sodium chloride buffer (Buffer B). DOC antigen (1-10 pg) suspended in buffer B was added directly on top of the sensor. The DOC was incubated for 10 minutes to 4 hours. Known Lyme positive plasma samples were subsequently analyzed on DOC immobilized sensors.

The recombinant protein, DOC, provides an efficient capture antigen that can bind antibodies against Bb (IgG, IgM or both). As mentioned above, “DOC” antigen consists of three epitopes—PepC10—immunogenic part of OspC, C-6-immunogenic part of VlsE and full-length DbpA combined in a recombinant protein. PepC-10 and DbpA bind IgM whereas C6 and DbpA bind IgG. Therefore, it is believed that the use of the “DOC” antigen will capture both circulating IgM and IgG.

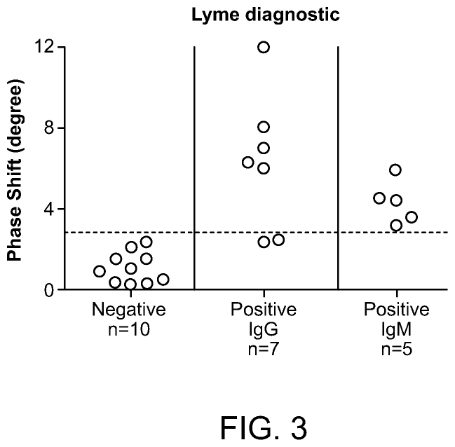

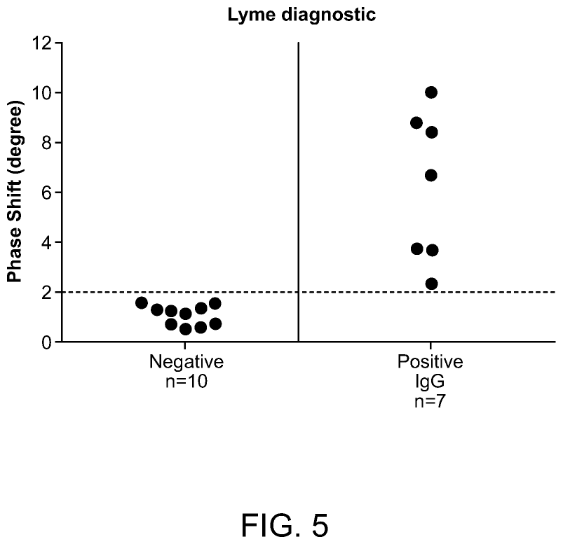

Plasma samples from healthy donors (n=11) were analyzed to establish the threshold/background cutoff value of the test. Preliminary data were collected using known Lyme disease positive plasma samples (IgG positive; n=7 and IgM positive; n=5) to evaluate the sensitivity of the sensor. Results indicate high sensitivity for IgM (100%), however, IgG shows an overlap with control samples as shown in FIG. 3 that shows the phase shift of the sensor with IgG and IgM positive plasma samples using the DOC immobilized SAW sensor. Therefore, to improve the sensitivity and selectivity of the assay method, a secondary antibody specific to human IgG (Anti-human-IgG) was used as second step to amplify the mass loading on the sensor that shows examples of affinity based strategies for the capture and enhanced sensitivity of Lyme disease detection by mass amplification of the SAW device. The secondary antibody use in this technique increases the sensitivity of sensor for screening Lyme IgG positive plasma and it was thus possible to detect all seven Lyme positive IgG plasmas as shown in FIG. 5, illustrating the phase shift of the sensor with secondary anti-IgG antibody cross absorbed with human IgM and IgA.

It is possible to increase the sensitivity of the acoustic sensors, and in an example the SAW sensors, by mass amplification. The circulating concentrations of the IgG and IgM vary in patients due to different immune responses and disease stages. As a point of care (POC) technology, the described sensor platform should work on finger stick blood, which is less than about 50 ul. Therefore, the described sensor platform should have high working sensitivity in the range of low picograms to femtograms range. To achieve this enhanced level of sensitivity, it is possible to employ an antibody (or their Fab fragments, or aptamers, etc.) in a sandwich format as shown in FIG. 4. Because the SAW sensor is mass-sensitive, the addition of a second antibody after the Lyme IgG and/or IgM has already been captured by the surface bio-coating adds additional mass to the sensor and thereby improves the sensitivity for any given analyte. More importantly, if the second antibody is itself tagged with a very much larger mass, for example, the ball shown in FIG. 3 as polystyrene or gold nanoparticles, the resultant increase in mass bound to the sensor can be many orders of magnitude greater than that of the original analyte or the second antibody itself.

FIG. 5 helps explain this example of affinity-based strategies for the capture and enhanced sensitivity of Lyme disease detection by mass amplification on a SAW device. For example, the mass of a single 200 nm polystyrene bead (2.51 femtograms) is nearly 4 orders of magnitude greater than that of an IgG antibody (0.00024 femtograms). Thus, the impact of the amplification strategy on analyte sensitivity is massive. For example, simple calculations, based upon the preliminary data in FIG. 3, suggest that with a sandwich approach, the SAW sensor could detect IgG and IgM in femtomolar range. Moreover, polystyrene beads can be substituted with high density metallic beads (e.g., gold) to gain even further increases in sensitivity. Thus, the use of a sandwich technique enhanced by mass amplification is a novel technique to dramatically augment the sensitivity for Lyme disease diagnosis. Moreover, mass amplification can be used with any analyte (large particles down to small molecules) for which specific pairs of antibodies or aptamers are available.

It is possible to use a mobile sensing device, including a mobile reader integrated with a mobile phone. Disposable cartridges could be used and data management transferred to a mobile phone that communicates and controls the reader via an on-phone USB port or connector. It is possible to include a dedicated software application on a reader and dedicated phone such as an Android phone to calculate any phase changes of a readout signal and translate phase-change values of degrees to an analyte concentration based on a calibration standard curve to transmit results wirelessly via WiFi or Bluetooth or other connector to a smart device as a mobile phone and perform quality control of any readers and test cartridges and display test results. Test results can be managed and connect to a reader via third party POC data management systems and interfaced to an electronic medical record (EMR) via laboratory information system (LIS). This could allow interfacing with a laboratory and hospital information systems (LIS/HIS) and wirelessly communicate real-time results. An integrated test cartridge could interface with various components. Separate cartridges could be used to test for IgM and IgG in an example.

Many modifications and other embodiments of the disclosure will come to the mind of one skilled in the art having the benefit of the teachings presented in the foregoing descriptions and the associated drawings. Therefore, it is to be understood that the disclosure is not to be limited to the specific embodiments disclosed, and that the modifications and embodiments are intended to be included within the scope of the dependent claims.

### Diagnostics

The present disclosure features diagnostic assays for the detection of polypeptides or antibodies that are correlated with infectious disease (e.g., MERS-COV, SARS-COV, SARS-COV-2, H1N1 influenza, Ebola, Lyme's disease, and the like). In one embodiment, levels of antibodies directed against S-protein, N-protein, E-protein, M-protein, and NSP1, NSP2, NSP3, NSP4, NSPS, NSP6, NSP7, NSP8, NSP9, NSP10, NSP12, NSP13, NSP14, NSP15, and/or NSP16 from SARS-COV-2 may be detected to assess presence or absence of infectious disease. S-protein, N-protein, E-protein, M-protein, and NSP1, NSP2, NSP3, NSP4, NSP5, NSP6, NSP7, NSP8, NSP9, NSP10, NSP12, NSP13, NSP14, NSP15, and/or NSP16 antibodies are measured in a subject sample to identify the presence of infectious disease, such as, for example, COVID-19.

The techniques herein may be used to measure levels of antibodies in any biological sample. Biological samples include tissue samples (e.g., cell samples, biopsy samples) and bodily fluids, including, but not limited to, saliva, blood, blood serum, and plasma.

In embodiments, a 2, 3, 4, 5, 6, 7, 8, 9, 10, 11, 12, 13, 14, 15, 16, 17, 18, 19, 20, 21, 22, 23, 24, or 25-fold change (increase or decrease) in the level of a detected antibody of the disclosure is indicative of the level of severity or time line of viral infection. Research has shown that there is a correlation between viral titers and severity of disease. The ability to detect viral titer and count directly without resorting to infective assays provides valuable information to the medical practitioner on the severity of disease and its length of prevalence. This also provides important information about the relative transmissibility of the virus. Importantly, the relative transmissibility, patient morbidity, and lethality of viruses (e.g. SARS-CoV-2) has been shown to correlate with viral titers and counts. (C. Henegan et al, March 2020, Center for Evidence Based Medicine, Oxford University).

In yet another embodiment, an expression profile that detects the presence of antibodies to two, three, or more SARS-COV-2 polypeptides correlates with an increased confidence interval of COVID19 positive test results. This embodiment of the instant disclosure is especially needed as extant SARS-COV-2 serological testing kits exhibit poor specificity.

The diagnostic methods described herein can also be used to monitor and manage progression or treatment of an infectious disease caused by, for example, MERS-COV, SARS-COV, SARS-COV-2, H1N1 influenza, Ebola, and the like.

### Diagnostic Kits

The disclosure provides kits for diagnosing or monitoring infectious disease (e.g., MERS-COV, SARS-COV, SARS-COV-2, H1N1 influenza, Ebola, and the like). In some embodiments, the kit comprises a sterile container which contains the binding agent; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, the kit is provided together with instructions for using the kit to characterize the infectious disease. The instructions will generally include information about the use of the composition for diagnosing a subject as having infectious disease or having a propensity to develop infectious disease. In other embodiments, the instructions include at least one of the following: description of the binding agent; warnings; indications; counter-indications; animal study data; clinical study data; and/or references. The instructions may be printed directly on the container (when present), or as a label applied to the container, or as a separate sheet, pamphlet, card, or folder supplied in or with the container.

### Subject Monitoring

The disease state or treatment of a subject having an infectious disease, or a propensity to develop an infectious disease can be monitored using the methods and compositions of the disclosure. Such monitoring may be useful, for example, in assessing the efficacy of a particular drug in a subject or in assessing disease progression. Therapeutics that increase or decrease the expression of a marker of the disclosure (e.g., S-protein, N-protein, E-protein, M-protein, and NSP1, NSP2, NSP3, NSP4, NSPS, NSP6, NSP7, NSP8, NSP9, NSP10, NSP12, NSP13, NSP14, NSP15, and NSP16) so as to reduce or eliminate the infectious disease are taken as particularly useful in the disclosure. Further, the kits of the instant disclosure are amenable to home use, for which there is a dire need during pandemics, such as that of the current SARS-CoV-2.

### Selection of a Treatment Method

Quantitative detection of viral load informs the success of any therapeutic intervention. Semi-quantitative detection of antibody triter will help to determine the success of the vaccine candidate development.

### EXAMPLES

The Examples described below broadly relate to detection of infectious disease.

### Example 1: A Method for Producing and Use of the Recombinant Antigen SSNs Consist of Different Immunogenic Epitopes of SARS-Cov-2 Virus for COVID19 Diagnosis

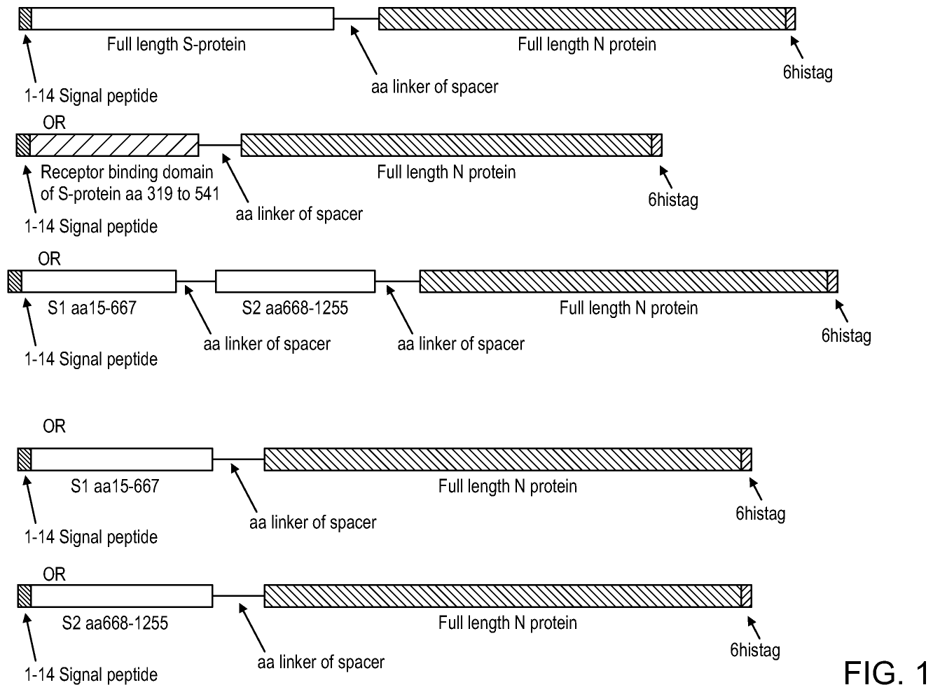

Novel composite proteins consisting of various combinations of SARS-CoV-2 surface protein, termed SSNx are made using recombinant technologies (SSN1 to SSNxx) and assayed by covalently attaching these antigens to the sensor surface using the binding processes described herein. FIG. 1 shows different example configurations of the SSN antigen for COVID19 diagnosis. Such SSNx proteins consist of a series of recombinant antigens synthesized using sequencing techniques and may consist of any number of the following recombinant proteins such as the full-length S and N protein or the receptor binding domain of S protein (amino acids 319-541) and the full length N-protein, 51 and S2 submit of S protein and the full length N-protein, or the 51 submit and the full length of the N-protein, or the S2 subunit of S protein and full length N-protein of SARS-Cov-2. Other combinations include full-length or subunits of any of the Non-Structural Proteins 1-16 in combination with the S, N, E and/or M proteins full length or subunit sequences. In the recombinant antigen, different epitopes are separated by an amino acid spacer or linker. Linkers may include for example, but not limited to, Serine and Glycine in combinations of 3-20 amino acids, Glycine repeats of 8-10 amino acids, and rigid linkers of (EAAAK)ₙ where n=1-3.

The sensor surface captures specific IgG and IgM antibodies present in infected patient plasma samples. Recombinant technology is used to prepare different combinations of antigenic epitopes to produce a series of SSN-antigens for the COVID19 diagnostic. Previous methods developed by the Applicant include recombinant antigen “DOC” which consisted of full length full-length DbpA, PepC10, C6, on the sensor surface to capture Borrelia (Bb) burgdorferi-specific IgG and IgM antibodies present in infected patient plasma samples with Lyme disease. The sensor platform for Lyme disease may become the POC method for detection of antibodies against Bb.

The platform described herein using SSN-antigen or similar recombinant antigens or mixtures of antigens can be used for SARS-CoV-2 serological diagnosis. It is very important to bind the antigen/s covalently having a proper orientation for binding with Bb specific antibodies from infected blood, serum or plasma. The surface of the SAW sensor described herein is metal (Aluminum) deposited on a crystal surface. Sections of the sensor also contain aluminum alternating with crystal. Various crystals can be used along with various crystal cuts. Nevertheless, all possible approaches regarding the use of SAW sensors or other biosensing modes for the detection of specific antibodies are based on the ability to decorate the sensor surface with an appropriate antigen, as discussed above. For the detection of specific antibodies (IgG and IgM), the sensor surface is decorated with the appropriate antigen material that can selectively capture the desired target specific IgG, IgM or both.

In some embodiments, the method and use of SSNx recombinant multiepitope antigen for diagnosis of COVID19 and method can be used to attach SSNx antigen on the SAW based metal or crystal sensor surface as a capturing molecule. However, the approach is not limited to antigens and can be adapted to immobilize other capture agents with primary amine group including but not limited to protein, protein fragments, antibody, antibody fragments, aptamers or nucleotide fragments, small molecules on the sensor surface. Other embodiments include a method that specifically enhances the detection sensitivity of the sensor.

According to the disclosure, the sensor may be coated with antibodies against virus surface protein or viral protein, allowing the sensor to detect the antigenic surfaces of the virions and provide a quantitative read of viral load or presence of virus protein in the sample. This may be facilitated by the generation of an affinity agent such as an antibody, aptamer or affirmer to the recombinant protein synthesized as noted above. The antibody may be placed on the sensor and nasal swab or other biological samples flown over the sensors. The specific binding of the antigens or virus particles to the antibodies coated on the sensor may then elicit an electronic change on the sensor as noted above.

### Example 2: A Method for Surface Activation and Derivatization Followed by the Bio-Coating of Aluminum

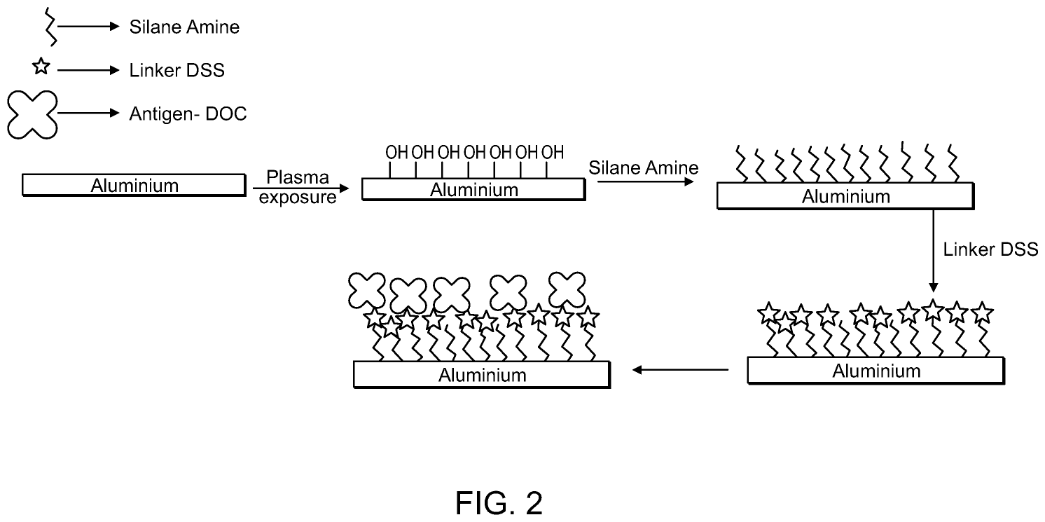

In some embodiments, the native Al and crystal surface was first activated by plasma cleaning (on the time scale of minutes to hours). The exposure of the sensor to plasma cleaning creates hydrophilic functional groups on AL and crystal surfaces that can be readily measured by evaluating the water contact angle. Contact angles significantly less than 90° are optimal for subsequent attachment of reagents to the activated surface. The activated surface was subsequently coated with a silane with amine functional group. The concentration of the Silane is important to ensure a monolayer and depends on the reaction conditions used. 1-10% of Silane in alcohol:water mixture (pH—4-6) was used for the coating. Coating was carried out for 30 minutes to 1 hr. Following the coating, the sensors were washed to remove excess unreacted silane from the activated Al surface. The coated devices were then dried under nitrogen gas and heated (100-130° C.) to create covalent bonds between the silane and aluminum surface. Next, disuccinimidyl suberate—a linker molecule—was covalently attached to the amine silane. The reaction was carried out at pH 6-7.5 using sodium phosphate—sodium chloride buffer (Buffer B). Then excess DSS was removed by washing and SNN antigen (1-10 μg) suspended in buffer was added directly to the sensor. The SSN suspension was then incubated for 30 minutes to 1 hr. After a thorough wash with saline, sensors were sequentially blocked using casein (2-10%) and polyvinyl alcohol to reduce non-specific binding. The SSN immobilized sensor is then ready for COVID diagnosis. FIG. 2 is a schematic representation of the bio-coating developed with an antigen as a preferred recombinant antigen as an epitope of a Lyme disease Borrelia species for selective capturing Bb specific IgG and IgM or both.

### Example 3: A Procedure for Increasing the Sensitivity of SAW Sensors by Mass Amplification

In some embodiments, the above method was followed by immobilizing DOC antigens on the SAW sensor. The recombinant protein, DOC, provided an efficient capture antigen to bind antibodies against Bb (IgG, IgM or both). As mentioned above, the “DOC” antigen consists of three epitopes—PepC10—immunogenic part of OspC, C-6-immunogenic part of VlsE and full-length DbpA combined in a recombinant protein. PepC-10 and DbpA bind IgM whereas C6 and DbpA bind IgG. Therefore, the use of the “DOC” antigen captured both circulating IgM and IgG. Plasma samples from healthy donors (n=11) were analyzed to establish the threshold/background cutoff value of the test. Preliminary data were collected using known LD positive plasma samples (IgG positive; n=7 and IgM positive; n=5) to evaluate the sensitivity of the sensor. Results indicated high sensitivity for IgM (100%), however IgG shows an over-lap with control samples (FIG. 3).

Therefore, to improve the sensitivity and selectivity of the assay method a secondary antibody specific to human IgG (Anti-human-IgG) was used as second step to amplify the mass loading on the sensor (FIG. 4). FIG. 4 shows examples of affinity bases strategies for the capture and enhanced sensitivity of Lyme disease detection by mass amplification on a SAW device. The secondary antibody used in the method significantly increased the sensitivity of sensor for screening Lyme IgG positive plasma such that all seven Lyme positive IgG plasmas were identified (FIG. 5).

The circulating concentrations of the IgG and IgM vary in patients due to different immune response and disease stage. As a point of care technology, the SAW platform must work on fingerstick blood which is <50 μl. Therefore, in some embodiments, the platform device requires working sensitivity in the low picograms to femtograms range. To achieve this enhanced level of sensitivity, a method that employs an antibody (or their Fab fragments, or aptamers, etc.) in a sandwich format was developed, as shown in FIG. 4. As the SAW sensor is mass-sensitive, the addition of a second antibody after the Lyme IgG and/or IgM captured by the surface bio-coating, adds additional mass to the sensor and thereby improves the sensitivity for any given analyte. Importantly, if the second antibody is itself tagged with a much larger mass (FIG. 3 red ball, e.g., a polystyrene or gold nanoparticles), the resultant increase in mass bound to the sensor can be many orders of magnitude greater than that of the original analyte or the second antibody itself.

For example, the mass of a single 200 nm polystyrene bead (2.51 femtograms) is nearly 4 orders of magnitude greater than that of an IgG antibody (0.00024 femtograms). Thus, the impact of the amplification strategy on analyte sensitivity is massive. For example, simple calculations, based upon the preliminary data in FIG. 4, suggest that with a sandwich approach, the SAW sensor could detect IgG and IgM in femtomolar range. Moreover, polystyrene beads can be substituted with high density metallic beads (e.g., gold) to gain even further increases in sensitivity. Thus, the use of a sandwich technique enhanced by mass amplification is a novel means to dramatically augment the sensitivity for COVID19 or Lyme disease diagnosis. Moreover, mass amplification can be used with any analyte (large particles down to small molecules) for which specific pairs of antibodies or aptamers are available.

### Example 4: A Method for Antigen Detection Using an Antibody to a Recombinant Protein

In some embodiments, specific recombinant antigens are developed and used to create antibodies which themselves are bound covalently to the substrate. Recombinant antigen-derived antibodies facilitate precise measurements of viral titer and viral protein presence from unprocessed clinical samples, which may contain low viral titer. Such antibodies can be used for example, in acoustic detection systems, such as SAW, as described herein.

### OTHER EMBODIMENTS

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A biosensor component, comprising:
a substrate; and
at least one antigen immobilized on the substrate, wherein said at least one antigen comprises an antigen or epitope of an infectious disease pathogen selected from the group consisting of a bacteria, a fungus, a parasite, and a virus.

2. The biosensor component according to claim 1, wherein said at least one antigen comprises, S-protein, N-protein, E-protein, M-protein, and NSP1, NSP2, NSP3, NSP4, NSP5, NSP6, NSP7, NSP8, NSP9, NSP10, NSP12, NSP13, NSP14, NSP15, NSP16, OspC, BmpA, VIsE(C6), DbpA, BBK19, OspA, RevA, Crasp2, BBK50, or portions thereof or combinations or fusions thereof.

3. The biosensor component according to claim 1, wherein said at least one antigen comprises a recombinant SARS-COV-2 antigen comprising a full-length S-protein linked to full-length N-protein, or the receptor binding domain of S-protein (aa 319-541) linked to the full length N-protein, or the 51 and/or S2 subunit of the S-protein linked to the full length N-protein, or 51 subunit of the S-protein linked to the full length N-protein, or the S2 subunit of S-protein linked to the full length N-protein of SARS-Cov-2, or a recombinant Lymes Disease antigen comprising a full-length DbpA protein fused to the C6 peptide of VIsE and the PEP10 peptide of OspC to capture IgM and IgG SARS-COV-2 or Borrelia burgdorferi specific antibodies.

4. The biosensor component according to claim 1, further comprising a metal coating on the substrate, wherein the metal is selected from the group consisting of aluminum, gold, and aluminum alloy, and any combination thereof.

5. The biosensor component according to claim 4, wherein the metal comprises aluminum.

6. The biosensor component according to claim 1, further comprising a linker binding the at least one antigen for covalent conjugation.

7. The biosensor component according to claim 6, wherein said linker comprises disuccinimidyl suberate.

8. The biosensor component according to claim 1, wherein said substrate comprises a piezoelectric substrate.

9. The biosensor component according to claim 1, further comprising an acoustic wave transducer.

10. The biosensor according to claim 9, wherein the acoustic wave transducer generates at least one of surface acoustic waves, bulk acoustic waves, Raleigh acoustic waves and Love waves.

11. The biosensor according to claim 1, wherein said substrate further comprises a pair of electrical components, at least one of the electrical components comprising an interdigital transducer.

12. A sensor for Point of Care diagnosis of an infectious disease, comprising:
a sensor housing;
a substrate carried by the sensor housing; and
at least one antigen immobilized on the substrate, wherein said at least one antigen comprises an antigen or epitope of an infectious disease pathogen selected from the group consisting of a bacteria, a fungus, a parasite, and a virus.

13. The sensor according to claim 12, wherein said at least one antigen comprises, S-protein, N-protein, E-protein, M-protein, and NSP1, NSP2, NSP3, NSP4, NSP5, NSP6, NSP7, NSP8, NSP9, NSP10, NSP12, NSP13, NSP14, NSP15, NSP16, OspC, BmpA, VIsE(C6), DbpA, BBK19, OspA, RevA, Crasp2, BBK50, or portions thereof or combinations or fusions thereof.

14. The sensor according to claim 12, wherein said at least one antigen comprises a recombinant SARS-COV-2 antigen comprising a full-length S-protein linked to full-length N-protein, or the receptor binding domain of S-protein (aa 319-541) linked to the full length N-protein, or the S1 and/or S2 subunit of the S-protein linked to the full length N-protein, or 51 subunit of the S-protein linked to the full length N-protein, or the S2 subunit of S-protein linked to the full length N-protein of SARS-Cov-2, or a recombinant Lymes Disease antigen comprising a full-length DbpA protein fused to the C6 peptide of VIsE and the PEP10 peptide of OspC to capture IgM and IgG SARS-COV-2 or Borrelia burgdorferi specific antibodies.

15. The sensor according to claim 12, further comprising a linker binding the at least one antigen for covalent conjugation.

16. The sensor according to claim 15, wherein said linker comprises disuccinimidyl suberate.

17. The sensor according to claim 12, wherein said substrate comprises a piezoelectric substrate.

18. The sensor according to claim 12, further comprising an acoustic wave transducer.

19. The sensor according to claim 18, wherein the acoustic wave transducer generates at least one of surface acoustic waves, bulk acoustic waves, Raleigh acoustic waves and Love waves.

20. The sensor according to claim 12, wherein said substrate further comprises a pair of electrical components, at least one of the electrical components comprising an interdigital transducer.

21. A method of fabricating a biosensor component, comprising:
exposing a substrate surface to plasma or gaseous cleaning to create on the substrate surface hydrophilic functional groups;
applying silane having an amine functional group onto the substrate surface to form a silane coating; and
exposing the silane coating to a buffer solution having a linker and an antigen, wherein said at least one antigen comprises an antigen or epitope of an infectious disease pathogen selected from the group consisting of a bacteria, a fungus, a parasite, and a virus.

22. The method according to claim 21, wherein said at least one antigen comprises, S-protein, N-protein, E-protein, M-protein, and NSP1, NSP2, NSP3, NSP4, NSP5, NSP6, NSP7, NSP8, NSP9, NSP10, NSP12, NSP13, NSP14, NSP15, NSP16, OspC, BmpA, VIsE(C6), DbpA, BBK19, OspA, RevA, Crasp2, BBK50, or portions thereof or combinations or fusions thereof.

23. The method according to claim 21, wherein said at least one antigen comprises a recombinant SARS-COV-2 antigen comprising a full-length S-protein linked to full-length N-protein, or the receptor binding domain of S-protein (aa 319-541) linked to the full length N-protein, or the S1 and/or S2 subunit of the S-protein linked to the full length N-protein, or S1 subunit of the S-protein linked to the full length N-protein, or the S2 subunit of S-protein linked to the full length N-protein of SARS-Cov-2, or a recombinant Lymes Disease antigen comprising a full-length DbpA protein fused to the C6 peptide of VIsE and the PEP10 peptide of OspC to capture IgM and IgG SARS-COV-2 or Borrelia burgdorferi specific antibodies.

24. The method according to claim 21, wherein said linker comprises disuccinimidyl suberate.

25. The method according to claim 21, wherein said substrate comprises a piezoelectric substrate.

26. The method according to claim 21, further comprising forming an acoustic wave transducer.

27. The method according to claim 26, wherein the acoustic wave transducer generates at least one of surface acoustic waves, bulk acoustic waves, Raleigh acoustic waves and Love waves.

28. The method according to claim 21, wherein said substrate further comprises a pair of electrical components, at least one of the electrical components comprising an interdigital transducer.

29. The biosensor component of claim 1, wherein the virus belongs to a virus family selected from the group consisting of coronavirus, rhinovirus, and influenza.

30. The biosensor component of claim 3, wherein the recombinant protein comprises an internal linker.

31. The biosensor component of claim 30, wherein the internal linker is selected from the group consisting of 3-20 amino acids of serine, glycine, or combinations thereof, 8-10 amino acids of glycine, and (EAAAK)ₙ, where n=1-3
